(19) United States
(12) Patent Application Publication
(10) Pub. No.: US 2011/0224681 A1
(43) Pub. Date: Sep. 15, 2011

(54) SYSTEM AND METHOD FOR MAKING AND USING A SPLITABLE LEAD INTRODUCER FOR AN IMPLANTABLE ELECTRICAL STIMULATION SYSTEM

(75) Inventor: Matthew Lee McDonald, Pasadena, CA (US)

(73) Assignee: Boston Scientific Neuromodulation Corporation, Valencia, CA (US)

(21) Appl. No.: 13/044,941

(22) Filed: Mar. 10, 2011

Related U.S. Application Data
(60) Provisional application No. 61/314,000, filed on Mar. 15, 2010.

(51) Int. Cl. A61B 19/00

(52) U.S. Cl. 606/129

(57) ABSTRACT

A lead introducer includes a split-release insertion needle configured and arranged for insertion into an epidural space of a patient. The split-release insertion needle has a proximal end, a distal end, and a longitudinal axis. The split-release insertion needle includes a plurality of body elements that laterally mate along the longitudinal axis of the split-release insertion needle. When the plurality of body elements are mated, the plurality of body elements define a lumen along the longitudinal axis of the split-release insertion needle. The lumen is configured and arranged to receive a distal end of a neurostimulation lead. A removable retaining member is disposed over at least a portion of each of the plurality of body elements. The plurality of body elements are configured and arranged to at least partially separate from one another when the retaining member is removed from the plurality of body elements.

### CROSS-REFERENCE TO RELATED APPLICATIONS

This application claims the benefit under 35 U.S.C. §119(e) of U.S. Provisional Patent Application Ser. No. 61/314,000 filed on Mar. 15, 2010, which is incorporated herein by reference.

### FIELD

The present invention is directed to the area of implantable electrical stimulation systems and methods of making and using the systems. The present invention is also directed to a lead introducer with a split-release insertion needle for facilitating insertion of an implantable electrical stimulation, as well as methods of making and using lead introducers, split-release insertion needles, and electrical stimulation leads.

### BACKGROUND

Implantable electrical stimulation systems have proven therapeutic in a variety of diseases and disorders. For example, spinal cord stimulation systems have been used as a therapeutic modality for the treatment of chronic pain syndromes. Peripheral nerve stimulation has been used to treat chronic pain syndrome and incontinence, with a number of other applications under investigation. Functional electrical stimulation systems have been applied to restore some functionality to paralyzed extremities in spinal cord injury patients.

Stimulators have been developed to provide therapy for a variety of treatments. A stimulator can include a control module (with a pulse generator), one or more leads, and an array of stimulator electrodes on each lead. The stimulator electrodes are in contact with or near the nerves, muscles, or other tissue to be stimulated. The pulse generator in the control module generates electrical pulses that are delivered by the electrodes to body tissue.

### BRIEF SUMMARY

In one embodiment, a lead introducer includes a split-release insertion needle configured and arranged for insertion into an epidural space of a patient. The split-release insertion needle has a proximal end, a distal end, and a longitudinal axis. The split-release insertion needle includes a plurality of body elements that laterally mate along the longitudinal axis of the split-release insertion needle. When the plurality of body elements are mated, the plurality of body elements define a lumen along the longitudinal axis of the split-release insertion needle. The lumen is configured and arranged to receive a distal end of a neurostimulation lead. A removable retaining member is disposed over at least a portion of each of the plurality of body elements. The plurality of body elements are configured and arranged to at least partially separate from one another when the retaining member is removed from the plurality of body elements.

In another embodiment, a method for implanting a neurostimulation lead into a patient includes inserting a lead introducer into the patient. The lead introducer includes a split-release insertion needle disposed in a removable retaining member. The split-release insertion needle includes a plurality of body elements laterally mated with one another along a longitudinal axis of the split-release insertion needle to define a lumen extending along a length of the split-release insertion needle. A distal end of a neurostimulation lead is inserted into the lumen of the splitable insertion needle. The neurostimulation lead includes a plurality of electrodes disposed along the distal end of the neurostimulation lead and a plurality of terminals disposed along at least one proximal end of the neurostimulation lead. The lead introducer is guided in proximity to a target stimulation location within the patient. The retaining member is removed from the plurality of body elements, thereby causing the at least two of the plurality of body elements to at least partially separate from one another along the longitudinal axis of the split-release insertion needle. The at least two body elements are removed from the neurostimulation lead, leaving at least the distal end of the neurostimulation lead implanted in the patient.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying drawings, wherein:

FIG. 1 is a schematic view of one embodiment of an electrical stimulation system, according to the invention;

FIG. 2A is a schematic view of one embodiment of a proximal portion of a lead and a control module of an electrical stimulation system, according to the invention;

FIG. 2B is a schematic view of one embodiment of a proximal portion of a lead and a lead extension of an electrical stimulation system, according to the invention;

FIG. 3A is a schematic longitudinal cross-sectional view of one embodiment of a lead introducer with a removable retaining member disposed over a split-release insertion needle, according to the invention;

FIG. 3B is a schematic transverse cross-sectional view of one embodiment of the lead introducer of FIG. 3A, according to the invention;

FIG. 4A is a schematic transverse cross-sectional view of one embodiment of the lead introducer of FIG. 3A having a split-release insertion needle with three body elements disposed in a removable retaining member, according to the invention;

FIG. 4B is a schematic transverse cross-sectional view of one embodiment of the lead introducer of FIG. 3A having a split-release insertion needle with four body elements disposed in a removable retaining member, according to the invention;

FIG. 5A is a schematic side view of one embodiment of the split-release insertion needle of FIG. 3A having body elements removably coupled together along corresponding interlocking features disposed on the body elements, according to the invention;

FIG. 5B is a schematic side view of another embodiment of the split-release insertion needle of FIG. 3A having body elements removably coupled together along corresponding interlocking features disposed on the body elements, according to the invention;

FIG. 5C is a schematic transverse cross-sectional view of one embodiment of the lead introducer of FIG. 3A having a split-release insertion needle with body elements that include corresponding interlocking features disposed on the body elements, according to the invention;

FIG. 6 is a schematic transverse cross-sectional view of one embodiment of the lead introducer of FIG. 3A having a split-release insertion needle with a plurality of body elements coupled together by a hinge, according to the invention;

FIG. 7 is a schematic side view of a distal end of an obturator disposed in a lumen of a longitudinal cross-sectional view of the lead introducer of FIG. 3A, according to the invention;

FIG. 8 is a schematic side view of a distal end of a lead disposed in a lumen of a longitudinal cross-sectional view of the lead introducer of FIG. 3A, according to the invention; and

FIG. 9 is a schematic overview of one embodiment of components of a stimulation system, including an electronic subassembly disposed within a control module, according to the invention.

### DETAILED DESCRIPTION

The present invention is directed to the area of implantable electrical stimulation systems and methods of making and using the systems. The present invention is also directed to a lead introducer with a split-release insertion needle for facilitating insertion of an implantable electrical stimulation, as well as methods of making and using lead introducers, split-release insertion needles, and electrical stimulation leads.

Suitable implantable electrical stimulation systems include, but are not limited to, a least one lead with one or more electrodes disposed on a distal end of the lead and one or more terminals disposed on one or more proximal ends of the lead. Leads include, for example, percutaneous leads, paddle leads, and cuff leads. Examples of electrical stimulation systems with leads are found in, for example, U.S. Pat. Nos. 6,181,969; 6,516,227; 6,609,029; 6,609,032; and 6,741,892; and U.S. Patent Applications Publication Nos. 2003/0114905, 2005/0165465, 2007/0150036; 2007/0161294; 2007/0219595; 2007/0239243; 2007/0150007; and 2008/0071320, and U.S. patent application Ser. No. 11/238,240, all of which are incorporated by reference.

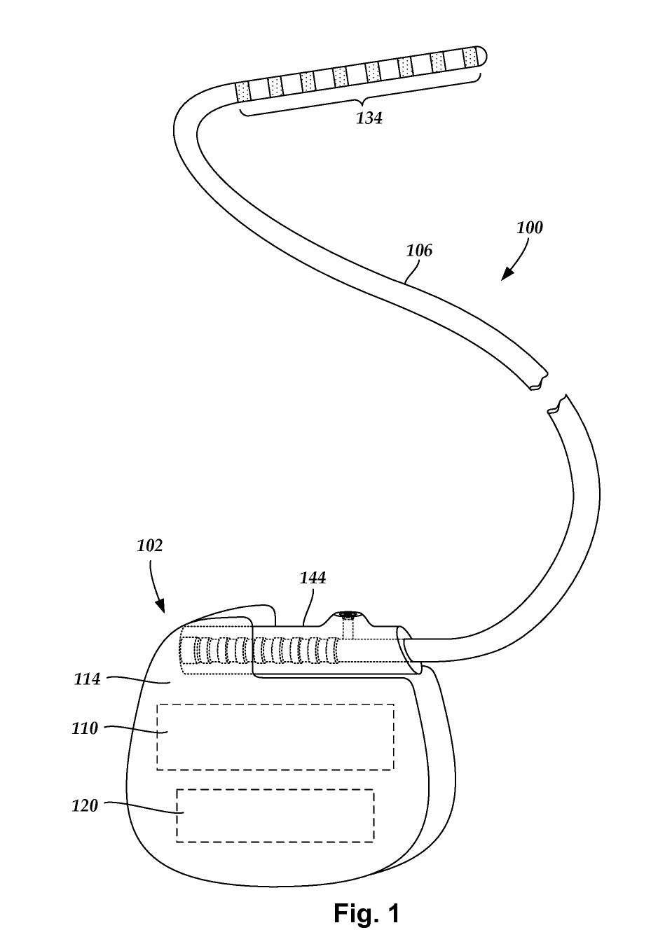

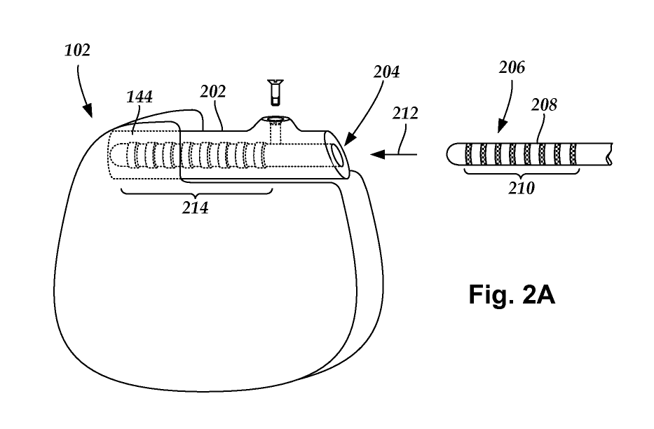

FIG. 1 illustrates schematically one embodiment of an electrical stimulation system 100. The electrical stimulation system includes a control module (e.g., a stimulator or pulse generator) 102 and at least one lead 106 coupled to the control module 102. Each lead 106 typically includes an array of electrodes 134. The control module 102 typically includes an electronic subassembly 110 and an optional power source 120 disposed in a sealed housing 114. The control module 102 typically includes a connector 144 (FIG. 2A, see also 222 and 250 of FIG. 2B) into which the proximal end of the one or more leads 106 can be plugged to make an electrical connection via conductive contacts on the control module 102 and terminals (e.g., 210 in FIGS. 2A and 236 of FIG. 2B) on each of the one or more leads 106. In at least some embodiments, a lead is isodiametric along a longitudinal length of the lead 106. In addition, one or more lead extensions 224 (see FIG. 2B) can be disposed between the one or more leads 106 and the control module 102 to extend the distance between the one or more leads 106 and the control module 102 of the embodiment shown in FIG. 1.

The electrical stimulation system or components of the electrical stimulation system, including one or more of the leads 106 and the control module 102, are typically implanted into the body of a patient. The electrical stimulation system can be used for a variety of applications including, but not limited to, brain stimulation, neural stimulation, spinal cord stimulation, muscle stimulation, and the like.

The electrodes 134 can be formed using any conductive, biocompatible material. Examples of suitable materials include metals, alloys, conductive polymers, conductive carbon, and the like, as well as combinations thereof. The number of electrodes 134 in the array of electrodes 134 may vary. For example, there can be two, four, six, eight, ten, twelve, fourteen, sixteen, or more electrodes 134. As will be recognized, other numbers of electrodes 134 may also be used.

The electrodes of one or more leads 106 are typically disposed in, or separated by, a non-conductive, biocompatible material such as, for example, silicone, polyurethane, polyetheretherketone (“PEEK”), epoxy, and the like or combinations thereof. The leads 106 may be formed in the desired shape by any process including, for example, molding (including injection molding), casting, and the like. The non-conductive material typically extends from the distal end of the one or more leads 106 to the proximal end of each of the one or more leads 106.

Terminals (e.g., 210 in FIGS. 2A and 236 of FIG. 2B) are typically disposed at the proximal end of the one or more leads 106 of the electrical stimulation system 100 for connection to corresponding conductive contacts (e.g., 214 in FIGS. 2A and 240 of FIG. 2B) in connectors (e.g., 144 in FIGS. 1-2A and 222 and 250 of FIG. 2B) disposed on, for example, the control module 102 (or to conductive contacts on a lead extension, an operating room cable, or an adaptor). Conductor wires (not shown) extend from the terminals (e.g., 210 in FIGS. 2A and 236 of FIG. 2B) to the electrodes 134. Typically, one or more electrodes 134 are electrically coupled to a terminal (e.g., 210 in FIGS. 2A and 236 of FIG. 2B). In at least some embodiments, each terminal (e.g., 210 in FIGS. 2A and 236 of FIG. 2B) is only connected to one electrode 134. The conductor wires may be embedded in the non-conductive material of the lead 106 or can be disposed in one or more lumens (not shown) extending along the lead 106. In some embodiments, there is an individual lumen for each conductor wire. In other embodiments, two or more conductor wires may extend through a lumen. There may also be one or more lumens (not shown) that open at, or near, the proximal end of the lead 106, for example, for inserting a stylet rod to facilitate placement of the lead 106 within a body of a patient. Additionally, there may also be one or more lumens (not shown) that open at, or near, the distal end of the lead 106, for example, for infusion of drugs or medication into the site of implantation of the one or more leads 106. In at least one embodiment, the one or more lumens may be flushed continually, or on a regular basis, with saline, epidural fluid, or the like. In at least some embodiments, the one or more lumens can be permanently or removably sealable at the distal end.

In at least some embodiments, leads are coupled to connectors disposed on control modules. In FIG. 2A, a lead 208 is shown configured and arranged for insertion to the control module 102. The connector 144 includes a connector housing 202. The connector housing 202 defines at least one port 204 into which a proximal end 206 of a lead 208 with terminals 210 can be inserted, as shown by directional arrow 212. The connector housing 202 also includes a plurality of conductive contacts 214 for each port 204. When the lead 208 is inserted into the port 204, the conductive contacts 214 can be aligned with the terminals 210 on the lead 208 to electrically couple the control module 102 to the electrodes (134 of FIG. 1) disposed at a distal end of the lead 208. Examples of connectors in control modules are found in, for example, U.S. Pat. No. 7,244,150 and U.S. patent application Ser. No. 11/532,844, which are incorporated by reference.

In FIG. 2B, a connector 222 is disposed on a lead extension 224. The connector 222 is shown disposed at a distal end 226 of the lead extension 224. The connector 222 includes a connector housing 228. The connector housing 228 defines at least one port 230 into which a proximal end 232 of a lead 234 with terminals 236 can be inserted, as shown by directional arrow 238. The connector housing 228 also includes a plurality of conductive contacts 240. When the lead 234 is inserted into the port 230, the conductive contacts 240 disposed in the connector housing 228 can be aligned with the terminals 236 on the lead 234 to electrically couple the lead extension 224 to the electrodes (134 of FIG. 1) disposed at a distal end (not shown) of the lead 234.

In at least some embodiments, the proximal end of a lead extension is similarly configured and arranged as a proximal end of a lead. The lead extension 224 may include a plurality of conductive wires (not shown) that electrically couple the conductive contacts 240 to a proximal end 248 of the lead extension 224 that is opposite to the distal end 226. In at least some embodiments, the conductive wires disposed in the lead extension 224 can be electrically coupled to a plurality of terminals (not shown) disposed on the proximal end 248 of the lead extension 224. In at least some embodiments, the proximal end 248 of the lead extension 224 is configured and arranged for insertion into a connector disposed in another lead extension. In other embodiments, the proximal end 248 of the lead extension 224 is configured and arranged for insertion into a connector disposed in a control module. As an example, in FIG. 2B the proximal end 248 of the lead extension 224 is inserted into a connector 250 disposed in a control module 252.

Some conventional percutaneous implantation techniques involve inserting a lead introducer, such as an epidural needle, into a patient. Once the lead introducer is inserted into the patient, a lead is inserted into a lumen of the lead introducer and guided in proximity to a target stimulation location. The lead is positioned at the target stimulation location and the lead introducer is removed from the patient, leaving the lead in place. Typically, the lead introducer is removed from the patient by sliding the lead introducer off the proximal end of the lead through the lumen of the lead introducer.

When a lead has a body that is not isodiametric along an entire length of the lead body, it may be difficult to completely slide the lead introducer off the proximal end of the lead. For example, when a proximal end of a lead body has a diameter that is larger than a distal end of the lead body, or when an oversized junction or adapter is disposed along the length of the lead body, the varying diameters along the length of the lead body may hinder, or even prevent, the lead introducer from sliding off the proximal end of the lead through the lumen of the lead introducer.

A splitable lead introducer (“lead introducer”) uses a split-release insertion needle (“insertion needle”) that enables the insertion needle to be separated from the lead without sliding the insertion needle off the proximal end of the lead through the lumen of the lead introducer. In at least some embodiments, the lead introducer includes an insertion needle formed from a plurality of body elements and a removable retaining member, such as heat shrink tubing, disposed over at least a portion of the insertion needle. When the retaining member is removed from the insertion needle, the body elements at least partially separate from one another, thereby enabling the body elements to separate from the lead. In at least some embodiments, when the retaining member is removed from the insertion needle, the body elements completely detach from one another.

In at least some embodiments, the body elements at least partially separate from one another along a longitudinal axis of the insertion needle. In at least some embodiments, the body elements separate from one another such that at least some of the plurality of body elements remain coupled together. In at least some embodiments, the body elements separate from one another such that at least some of the body elements completely detach from one another. When the body elements are separated (either partially or fully) from one another, the body elements may be removed from the patient, leaving the lead in place. In at least some embodiments, when the body elements are separated (either partially or fully) from one another, the body elements may be removed from the patient without sliding the insertion needle off the proximal end of the lead through the lumen of the lead introducer.

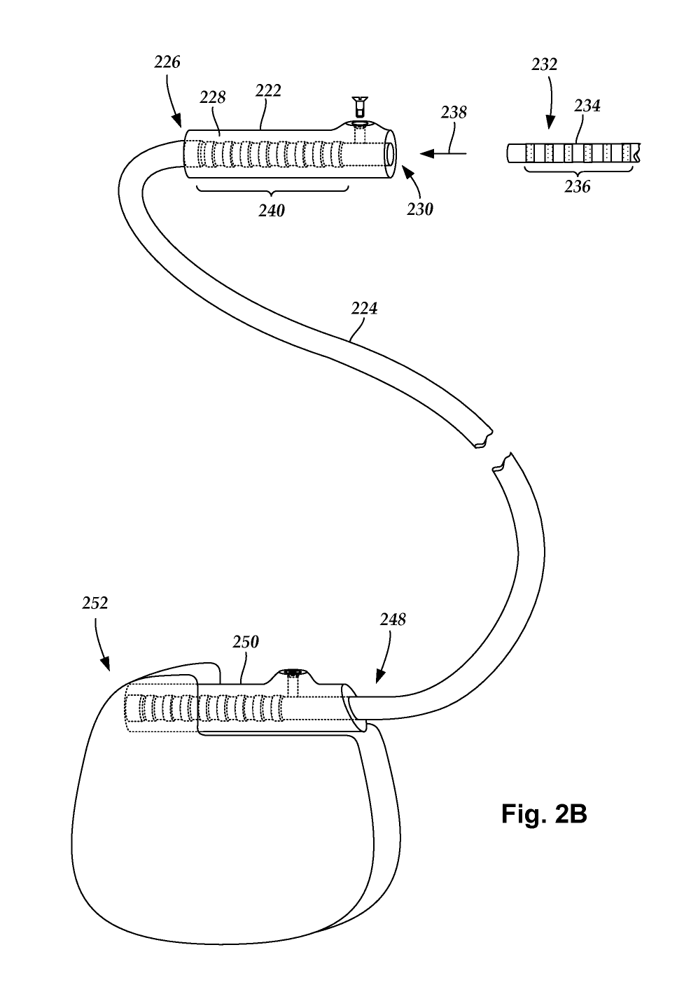

FIG. 3A is a schematic longitudinal cross-sectional view of one embodiment of a lead introducer 300 that includes an insertion needle 302 and a removable retaining member 304 disposed over the insertion needle 302. It will be understood that the components of FIG. 3A are not drawn to scale. For example, the thickness of the remaining member 304 is shown with an exaggerated thickness, for clarity of illustration.

FIG. 3B is a schematic transverse cross-sectional view of the lead introducer 300. The insertion needle 302 includes a proximal end 306, a distal end 308, and a longitudinal axis 310 (shown by a two-headed arrow). The insertion needle 302 also includes a plurality of body elements 312a and 312b mated together to define a lumen 316. In at least some embodiments, the body elements 312a and 312b are mated along the longitudinal axis 310 of the insertion needle 302. In at least some embodiments, the lumen 316 extends along the longitudinal axis 310. In at least some embodiments, the lumen 316 extends along the longitudinal axis 310 from the proximal end 306 to the distal end 308 of the insertion needle 302. In at least some embodiments, the lumen 316 extends from a proximal aperture 318 at the proximal end 306. In at least some embodiments, the lumen 316 extends from a distal aperture 320 at the distal end 308.

In at least some embodiments, the proximal end 308 includes a proximal hub 322. In at least some embodiments, the lumen 316 is in fluid communication with a luer fitting 324. In at least some embodiments, the luer fitting 324 is disposed on the proximal hub 322. In at least some embodiments, the luer fitting 324 is configured and arranged to receive a syringe. In at least some embodiments, fluid (e.g., saline solution, air, or the like) may then be introduced or removed through the luer fitting 324 to check for precise positioning of the lead introducer 300, for example, in an epidural space of the patient.

The retaining member 304 may be formed from any thermoplastic material suitable for implantation including, for example, polyester, polyolefin, one or more fluoropolymers (such as fluorinated ethylene propylene, polytetrafluoroethylene, polyvinylidene fluoride, or the like or combinations thereof), polyvinyl chloride, polychloroprene, silicone elastomer, or the like or combinations thereof.

In at least some embodiments, the retaining member 304 is disposed over at least a portion of an outer surface of the insertion needle 302. In at least some embodiments, the retaining member 304 is disposed substantially entirely over the outer surface of the insertion needle 302 distal to the proximal hub 322. In at least some embodiments, the retaining member 304 is disposed entirely over the outer surface of the insertion needle 302. In at least some embodiments, the retaining member 304 forms a watertight seal along the lumen 316 of the insertion needle 302. In at least some embodiments, the retaining member 304 provides a pressure-tight seal for the lumen 316. Providing the pressure-tight seal may enable a loss-of-resistance technique to be performed to ensure entry into the epidural space, as mentioned above.

The body elements 312a and 312b of the insertion needle 302 are formed from one or more rigid materials suitable for implantation (e.g., one or more metals, alloys, rigid plastics, or the like). In some embodiments, each of the body elements 312a and 312b are formed from the same material(s). In other embodiments, at least one of the body elements 312a and 312b is formed from one or more materials that are different from at least another one of the body elements 312a and 312b. The insertion needle 302 is formed from a material that is rigid enough to enable the insertion needle 302 to be used to guide the lead introducer 300 within a patient.

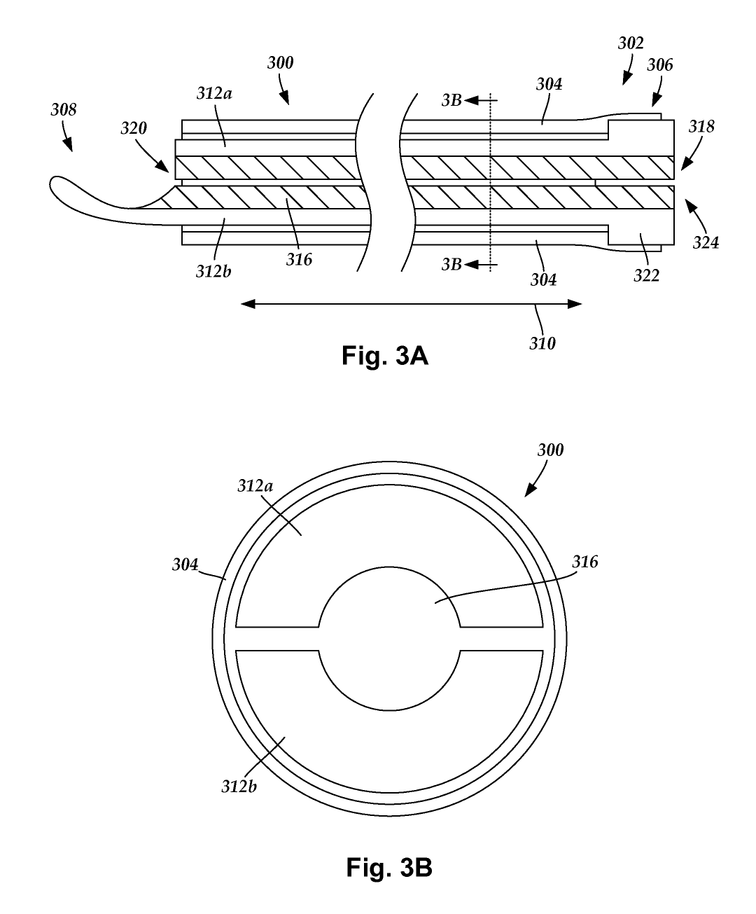

In at least some embodiments, the lateral circumference of the insertion needle 302 is no greater than sixteen-gauge, fifteen-gauge, fourteen-gauge, thirteen-gauge, or twelve-gauge. Any suitable number of body elements 312a and 312b may be used to form the insertion needle 302 including, for example, two, three, four, five, six, seven, eight, nine, ten, or more body elements. For example, FIGS. 4A-4B show several examples of the lead introducer 300 with different numbers of body elements. FIG. 4A is a schematic transverse cross-sectional view of one embodiment of the lead introducer 300 that includes three body elements 412a, 412b, and 412c disposed in the retaining member 304 to form the lumen 316. FIG. 4B is a schematic transverse cross-sectional view of one embodiment of the lead introducer 300 that includes four body elements 422a, 422b, 422c, and 422d disposed in the retaining member 304 to form the lumen 316.

In at least some embodiments, the body elements 312a and 312b include one or more interlocking features to maintain alignment of the body elements 312a and 312b within the retaining member 304. In at least some embodiments, the interlocking features maintain alignment of the body elements 312a and 312b along the longitudinal axis 310 of the insertion needle, particularly when the retaining member 304 is present. This can reduce, or even prevent, longitudinal sliding of the body elements 312a and 312b with respect to one another.

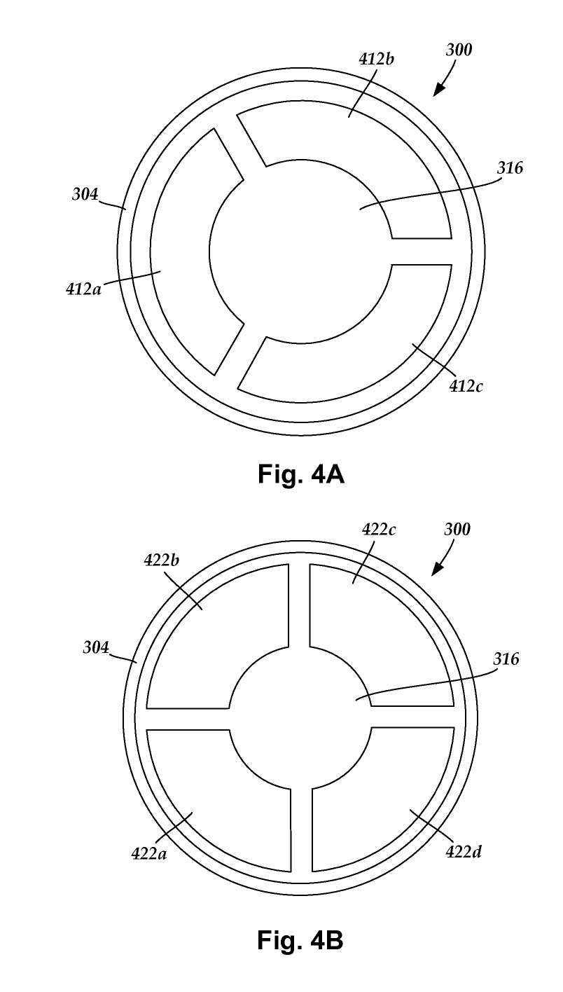

FIG. 5A and FIG. 5B are schematic side views of two embodiments of body elements 312a and 312b removably coupled together along corresponding interlocking features. Any suitable interlocking shapes may be used for the interlocking features. For example, FIG. 5A shows rectangular interlocking features 502a and 502b disposed on the body elements 312a and 312b, respectively. FIG. 5B shows rolling interlocking features 512a and 512b disposed on the body elements 312a and 312b, respectively. It will be understood that the body elements 312a and 312b may include any suitable number of interlocking features including, for example, one, two, three, four, five, six, seven, eight, nine, ten, or more interlocking features. It will be understood that there may be additional numbers of interlocking features.

In at least some embodiments, the interlocking features maintain alignment of the body elements 312a and 312b along an axis transverse to the longitudinal axis 310 of the insertion needle 302. FIG. 5C is a schematic transverse cross-sectional view of corresponding interlocking features 522a and 522b disposed on body elements 312a and 312b, respectively. The interlocking features 522a and 522b are configured and arranged to maintain alignment of the body elements 312a and 312b along an axis transverse to the longitudinal axis 310 of the insertion needle 302.

The interlocking features may be formed into any corresponding shapes (e.g., rectangular, triangular, trapezoidal, rolling, oval, or the like) suitable to interlock the body elements 312a and 312b to maintain alignment of the body elements 312a and 312b. In at least some embodiments, the interlocking features are shaped such that the body elements 312a and 312b are separatable from one another when not being bound by the retaining member 304.

The interlocking features may be disposed anywhere along the longitudinal axis 310 of the insertion needle 302. In some embodiments, the interlocking features extend along the entire longitudinal axis 310 of the insertion needle 302. In other embodiments, the interlocking features extend along one or more portions of the longitudinal axis 310 of the insertion needle 302. In at least some embodiments, the interlocking features are disposed at the proximal end 306 of the insertion needle 302. In at least some embodiments, the interlocking features are disposed at the distal end 308 of the insertion needle 302.

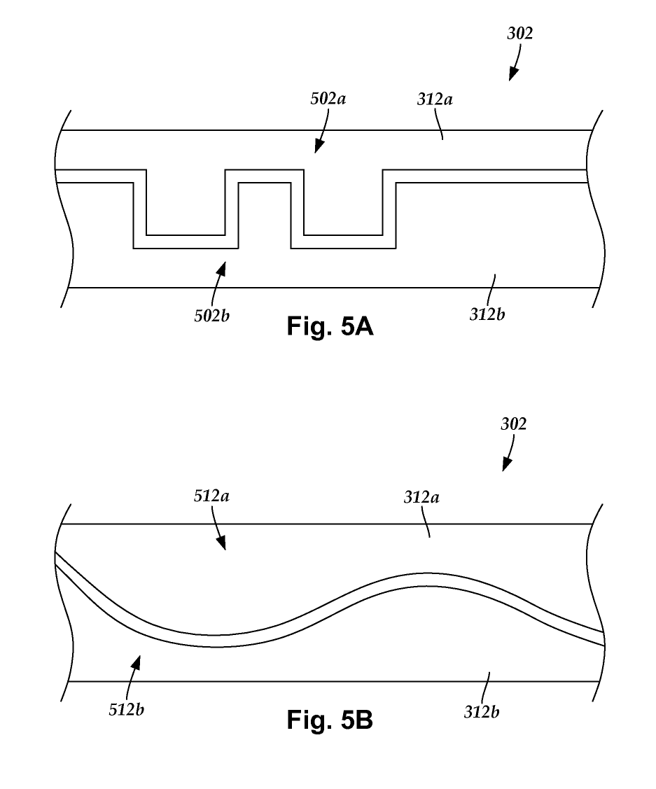

In at least some embodiments, at least some of the body elements are hinged together. FIG. 6 is a schematic transverse cross-sectional view of the body elements 312a and 312b disposed in the retaining member 304. The body elements 312a and 312b are coupled to each other via a hinge 602. In at least some embodiments, the hinge 602 is formed as a region of material that is thinner than surrounding materials, thereby forming a region that preferentially bends or folds to open. In at least some embodiments, the hinge 602 is formed as a region of material that is more flexible than surrounding materials, thereby forming a region that preferentially bends or folds to open.

In at least some embodiments, when the retaining member 304 is removed, the body elements 312a and 312b split apart such that the body elements 312a and 312b remain coupled together via the hinge 602. In at least some embodiments, the hinge is spring-loaded such that, when the retaining member 304 is removed, the body elements 312a and 312b split apart without applying an external force to the body elements 312a and 312b. In at least some embodiments, when the retaining member 304 is removed, the body elements 312a and 312b split apart when the body elements 312a and 312b are removed from the patient. In at least some embodiments, the hinge 602 opens at least as wide as a diameter of the lumen 316 of the insertion needle 302.

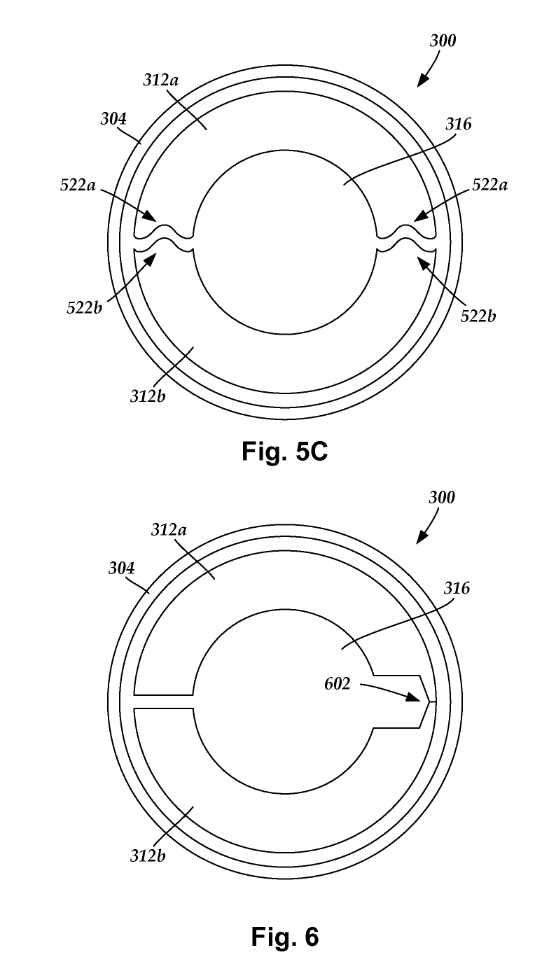

In at least some embodiments, the lumen 316 is configured and arranged to receive an obturator. FIG. 7 is a schematic side view of a distal end of an obturator 702 disposed in the lumen 316 of a longitudinal cross-sectional view of the lead introducer 300. In at least some embodiments, the obturator 702 is rigid. In at least some embodiments, the obturator 702 is rigid enough to be used to guide the lead introducer 300 within a patient. In at least some embodiments, the obturator 702 extends beyond the distal aperture 320 at the distal end 308 of the insertion needle 302. In at least some embodiments, the obturator 702 has a blunt tip to prevent coring of patient tissue during insertion of the lead introducer 300 into a patient.

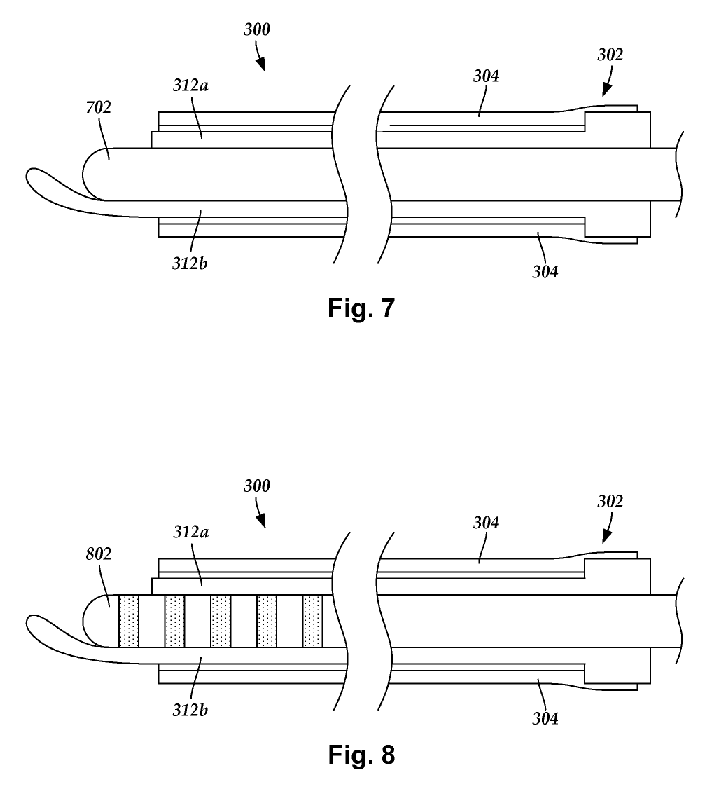

In at least some embodiments, the lumen 316 is configured and arranged to receive a distal end of a lead when the obturator 330 is not disposed in the lumen 316. FIG. 8 is a schematic side view of a distal end of a lead 802 disposed in the lumen 316 of a longitudinal cross-sectional view of the lead introducer 300. In some embodiments, the lead 802 has an isodiametric lead body. In other embodiments, the lead 802 has a non-isodiametric lead body. In at least some embodiments, the lead 802 includes one or more elements disposed along the length of the lead 802 which have a longitudinal cross-sectional shape or size that is different from at least one other portion of the lead 802. In at least some embodiments, the distal end of the lead 802 has a transverse shape that is similar to a transverse shape of the lumen 316.

In at least some embodiments, the lead 802 may be inserted into a patient using the lead introducer 300. In at least some embodiments, the lead introducer 300 is inserted into a patient and guided in proximity to a target stimulation location. In at least some embodiments, the insertion needle 302 is rigid enough to used to guide the lead introducer 300. In other embodiments, the obturator 702 is inserted into the lumen 316 and is used to guide the lead introducer 300 in proximity to the target stimulation location, and then removed. Once the lead introducer 300 is in proximity to a target stimulation location, the positioning of the lead introducer 300 may be checked. For example, to confirm that the lead introducer 300 is disposed in an epidural space of the patient. The positioning of the lead introducer 300 may be checked in any suitable manner, such as introducing or removing fluid through the luer fitting 324, imaging (e.g., via fluoroscopy, or the like) the patient with or without introducing one or more contrast agents into the patient, or using the electrodes of the lead (or another insertable stimulation device) to stimulate surrounding tissue.

In at least some embodiments, at least one of the retaining member 304 or the insertion needle 302 includes one or more radiopaque materials, for example, barium sulfate and bismuth subcarbonate, and the like or combinations thereof, that are incorporated into the lead introducer 300 to facilitate implantation of the lead 802 through the use of one or more medical imaging techniques, such as fluoroscopy. In at least some embodiments, once the lead introducer 300 is positioned in the epidural space in proximity to the target stimulation location, the distal end of the lead 802 may be inserted into the lumen 316 of the insertion needle 302.

Once the distal end of the lead 802 has been guided to the target stimulation location, the retaining member 304 may be removed from the lead introducer 300. In at least some embodiments, the retaining member 304 may be removed from the lead introducer 300 by rolling the retaining member 304 along the longitudinal axis 310 of the insertion needle 302. In at least some embodiments, the retaining member 304 may be removed from the lead introducer 300 by sliding the retaining member 304 along the longitudinal axis 310 of the insertion needle 302. In at least some embodiments, the retaining member 304 may be removed from the lead introducer 300 by rolling and sliding the retaining member 304 along the longitudinal axis 310 of the insertion needle 302. In at least some embodiments, an underside of the retaining member 304 includes a lubricious coating to facilitate sliding of the retaining member 304 along the longitudinal axis of the insertion needle 302.

It will be understood that the retaining member 304 can be removed in any convenient manner. For example, in at least some embodiments, the retaining member 304 may be torn into two or more pieces, or otherwise cut and removed. In at least some embodiments, the retaining member 304 has scored lines or areas where it preferentially tears along a certain direction.

Once the retaining member 304 is removed from the insertion needle 302, the retaining member 304 may be extracted from the patient. In at least some embodiments, once the retaining member 304 is removed from the insertion needle 302, the body elements at least partially split apart from one another to separate the insertion needle 302 from the lead 802. In at least some embodiments, the removal of the retaining member 304 causes the body elements to separate from the lead 802 without application of an external force, such as by a user of the lead introducer 300. In at least some other embodiments, when the retaining member 304 is removed, the body elements split apart when an external force is applied to the body elements 312a and 312b (e.g., when the body elements are removed from the patient).

Once the retaining member 304 is removed from the insertion needle 302, the body elements of the insertion needle 302 may be extracted from the patient. In at least some embodiments, each of the body elements may be extracted at once. In at least some embodiments, the body elements may be individually extracted from the patient. Preferably, the body elements of the insertion needle 302 may be extracted from the patient without moving the lead 802 from the target stimulation location. In at least some embodiments, the body elements of the insertion needle 302 may be extracted from the patient without passing the lumen 316 of the insertion needle 302 along the lead 802.

Once the lead 802 is positioned at the target stimulation site, the lead 802 can be coupled to a control module (e.g., 102 of FIG. 1) and implanted using well-known techniques, for example, using one or more using tunneling straws placed in passageways underneath patient skin with bores that are sized large enough to receive the lead 802. In at least some embodiments, the lead 802 can be coupled to a connector of a control module, as shown in FIG. 3. In other embodiments, the lead 802 can be coupled to one or more other devices, including an adaptor, a lead extension, an operating room cable, or the like or combinations thereof.

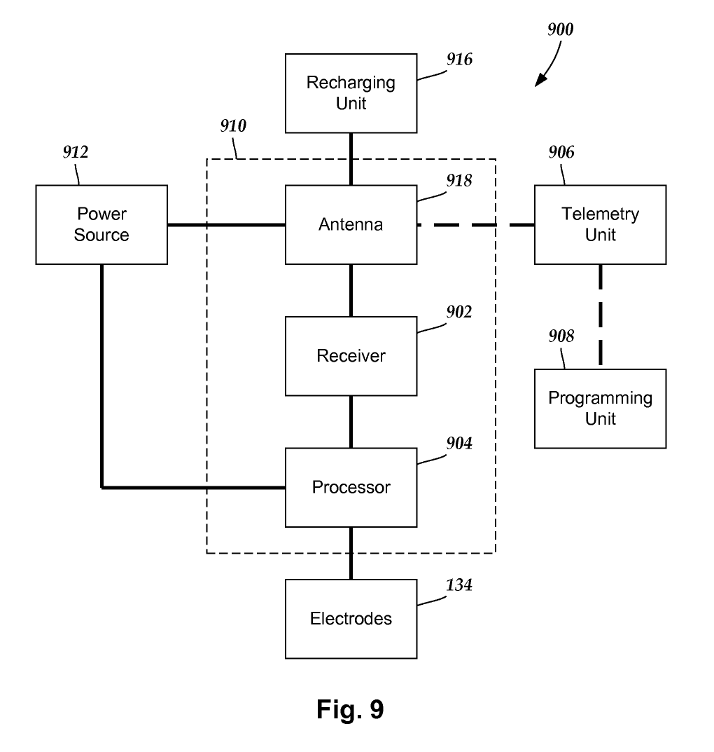

FIG. 9 is a schematic overview of one embodiment of components of an electrical stimulation system 900 including an electronic subassembly 910 disposed within a control module. It will be understood that the electrical stimulation system can include more, fewer, or different components and can have a variety of different configurations including those configurations disclosed in the stimulator references cited herein.

Some of the components (for example, power source 912, antenna 918, receiver 902, and processor 904) of the electrical stimulation system can be positioned on one or more circuit boards or similar carriers within a sealed housing of an implantable pulse generator, if desired. Any power source 912 can be used including, for example, a battery such as a primary battery or a rechargeable battery. Examples of other power sources include super capacitors, nuclear or atomic batteries, mechanical resonators, infrared collectors, thermally-powered energy sources, flexural powered energy sources, bioenergy power sources, fuel cells, bioelectric cells, osmotic pressure pumps, and the like including the power sources described in U.S. Patent Application Publication No. 2004/0059392, incorporated herein by reference.

As another alternative, power can be supplied by an external power source through inductive coupling via the optional antenna 918 or a secondary antenna. The external power source can be in a device that is mounted on the skin of the user or in a unit that is provided near the user on a permanent or periodic basis.

If the power source 912 is a rechargeable battery, the battery may be recharged using the optional antenna 918, if desired. Power can be provided to the battery for recharging by inductively coupling the battery through the antenna to a recharging unit 916 external to the user. Examples of such arrangements can be found in the references identified above.

In one embodiment, electrical current is emitted by the electrodes 134 on the paddle or lead body to stimulate nerve fibers, muscle fibers, or other body tissues near the electrical stimulation system. A processor 904 is generally included to control the timing and electrical characteristics of the electrical stimulation system. For example, the processor 904 can, if desired, control one or more of the timing, frequency, strength, duration, and waveform of the pulses. In addition, the processor 904 can select which electrodes can be used to provide stimulation, if desired. In some embodiments, the processor 904 may select which electrode(s) are cathodes and which electrode(s) are anodes. In some embodiments, the processor 904 may be used to identify which electrodes provide the most useful stimulation of the desired tissue.

Any processor can be used and can be as simple as an electronic device that, for example, produces pulses at a regular interval or the processor can be capable of receiving and interpreting instructions from an external programming unit 908 that, for example, allows modification of pulse characteristics. In the illustrated embodiment, the processor 904 is coupled to a receiver 902 which, in turn, is coupled to the optional antenna 918. This allows the processor 904 to receive instructions from an external source to, for example, direct the pulse characteristics and the selection of electrodes, if desired.

In one embodiment, the antenna 918 is capable of receiving signals (e.g., RF signals) from an external telemetry unit 906 which is programmed by a programming unit 908. The programming unit 908 can be external to, or part of, the telemetry unit 906. The telemetry unit 906 can be a device that is worn on the skin of the user or can be carried by the user and can have a form similar to a pager, cellular phone, or remote control, if desired. As another alternative, the telemetry unit 906 may not be worn or carried by the user but may only be available at a home station or at a clinician's office. The programming unit 908 can be any unit that can provide information to the telemetry unit 906 for transmission to the electrical stimulation system 900. The programming unit 908 can be part of the telemetry unit 906 or can provide signals or information to the telemetry unit 906 via a wireless or wired connection. One example of a suitable programming unit is a computer operated by the user or clinician to send signals to the telemetry unit 906.

The signals sent to the processor 904 via the antenna 918 and receiver 902 can be used to modify or otherwise direct the operation of the electrical stimulation system. For example, the signals may be used to modify the pulses of the electrical stimulation system such as modifying one or more of pulse duration, pulse frequency, pulse waveform, and pulse strength. The signals may also direct the electrical stimulation system 900 to cease operation, to start operation, to start charging the battery, or to stop charging the battery. In other embodiments, the stimulation system does not include an antenna 918 or receiver 902 and the processor 904 operates as programmed.

Optionally, the electrical stimulation system 900 may include a transmitter (not shown) coupled to the processor 904 and the antenna 918 for transmitting signals back to the telemetry unit 906 or another unit capable of receiving the signals. For example, the electrical stimulation system 900 may transmit signals indicating whether the electrical stimulation system 900 is operating properly or not or indicating when the battery needs to be charged or the level of charge remaining in the battery. The processor 904 may also be capable of transmitting information about the pulse characteristics so that a user or clinician can determine or verify the characteristics.

The above specification, examples and data provide a description of the manufacture and use of the composition of the invention. Since many embodiments of the invention can be made without departing from the spirit and scope of the invention, the invention also resides in the claims hereinafter appended.

What is claimed as new and desired to be protected by Letters Patent of the United States is:

1. A lead introducer comprising:
a split-release insertion needle configured and arranged for insertion into an epidural space of a patient, the split-release insertion needle having a proximal end, a distal end, and a longitudinal axis, the split-release insertion needle comprising a plurality of body elements that laterally mate along the longitudinal axis of the split-release insertion needle, wherein when the plurality of body elements are mated, the plurality of body elements define a lumen along the longitudinal axis of the split-release insertion needle, wherein the lumen is configured and arranged to receive a distal end of a neurostimulation lead; and
a removable retaining member disposed over at least a portion of each of the plurality of body elements;
wherein the plurality of body elements are configured and arranged to at least partially separate from one another when the retaining member is removed from the plurality of body elements.

2. The lead introducer of claim 1, wherein a one of the plurality of body elements comprises a first interlocking feature and another of the plurality of body elements comprises a second interlocking feature that corresponds to the first interlocking feature such that when the plurality of body elements mate along the longitudinal axis of the split-release insertion needle, the interlocking features maintain alignment of the plurality of body elements along at least one of the longitudinal axis of the split-release insertion needle or an axis transverse to the longitudinal axis of the split-release insertion needle.

3. The lead introducer of claim 1, wherein the split-release insertion needle comprises two, three, or four body elements.

4. The lead introducer of claim 1, wherein the plurality of body elements are configured and arranged to separate from one another such that at least one of the plurality of body elements completely separates from the other body elements when the retaining member is removed from the plurality of elements.

5. The lead introducer of claim 1, wherein the plurality of body elements are configured and arranged to separate from one another when the retaining member is removed from the plurality of elements such that at least two of the plurality of body elements remain at least partially attached to one another.

6. The lead introducer of claim 5, wherein the split-release insertion needle further comprises a hinge coupling two of the plurality of body elements to one another.

7. The lead introducer of claim 1, wherein the retaining member is heat shrink tubing.

8. The lead introducer of claim 1, wherein the retaining member is disposed over an entire length of the split-release insertion needle.

9. The lead introducer of claim 1, wherein the retaining member provides a watertight seal within the lumen of the split-release insertion needle.

10. The lead introducer of claim 1, wherein the retaining member is configured and arranged to roll up along or to slide along the longitudinal axis of the split-release insertion needle.

11. The lead introducer of claim 1, wherein the retaining member is configured and arranged to be torn into a plurality of pieces or split along a longitudinal axis of the retaining member.

12. An insertion kit comprising:
the lead introducer of claim 1;
a neurostimulation lead with a distal end configured and arranged for implantation into a patient, the neurostimulation lead comprising
a lead body having a distal end and a proximal end,
a plurality of electrodes disposed at the distal end of the lead body,
a plurality of terminals disposed at the proximal end of the lead body, and
a plurality of conductive wires coupling the plurality of electrodes electrically to the plurality of terminals; and
wherein the lumen of the split-release insertion needle is configured and arranged to receive the distal end of the lead body.

13. The insertion kit of claim 12, wherein the lead body is insertable into the lumen of the split-release insertion needle such that after insertion into the lumen, the lead body is separatable from the split-release insertion needle without sliding the lead body axially along the lumen of the split-release insertion needle.

14. The insertion kit of claim 12, wherein the lead body is non-isodiametric.

15. An electrical stimulation system comprising:
the insertion kit of claim 12;
a control module configured and arranged to electrically couple to the proximal end of the lead body, the control module comprising
a housing, and
an electronic subassembly disposed in the housing; and
a connector for receiving the neurostimulation lead, the connector comprising
a connector housing defining a port for receiving the proximal end of the lead body, and
a plurality of connector contacts disposed in the connector housing, the connector contacts configured and arranged to couple to at least one terminal disposed at the proximal end of the lead body.

16. The electrical stimulation system of claim 15, further comprising a lead extension having a proximal end and a distal end, the connector disposed on the distal end of the lead extension.

17. The electrical stimulation system of claim 16, wherein the proximal end of the lead extension is configured and arranged for insertion into another connector.

18. A method for implanting a neurostimulation lead into a patient, the method comprising:
inserting a lead introducer into the patient, the lead introducer comprising a split-release insertion needle disposed in a removable retaining member, the split-release insertion needle comprising a plurality of body elements laterally mated with one another along a longitudinal axis of the split-release insertion needle to define a lumen extending along a length of the split-release insertion needle;
inserting into the lumen of the splitable insertion needle a distal end of a neurostimulation lead, the neurostimulation lead comprising a plurality of electrodes disposed along the distal end of the neurostimulation lead and a plurality of terminals disposed along at least one proximal end of the neurostimulation lead;
guiding the lead introducer in proximity to a target stimulation location within the patient;
removing the retaining member from the plurality of body elements, thereby causing the at least two of the plurality of body elements to at least partially separate from one another along the longitudinal axis of the split-release insertion needle; and
removing the at least two body elements from the neurostimulation lead, leaving at least the distal end of the neurostimulation lead implanted in the patient.

19. The method of claim 18, wherein removing the retaining member from the at least two body elements comprises rolling up or sliding the removable retaining member along the longitudinal axis of the split-release insertion needle.

20. The method of claim 18, wherein removing the retaining member from the at least two body elements comprises tearing up the retaining member into a plurality of pieces or splitting the retaining member along a longitudinal axis of the retaining member.
